(19) United States
(12) Patent Application Publication
(10) Pub. No.: US 2015/0374405 A1
(43) Pub. Date: Dec. 31, 2015

(54) SUCTION CATHETER

(71) Applicant: Norikata TAKUMA, Kokubunji City (JP)

(72) Inventor: Norikata TAKUMA, Kokubunji City (JP)

(21) Appl. No.: 14/579,812

(22) Filed: Dec. 22, 2014

(30) Foreign Application Priority Data
Jun. 27, 2014 (JP) 2014-132363

(51) Int. Cl. A61B 17/3207

(52) U.S. Cl. CPC A61B 17/320758; A61B 17/320783; A61B 17/3207; A61B 2017/320733; A61B 2017/320766; A61B 2017/320775; A61B 2017/00778

(57) ABSTRACT

In a suction catheter to cut an object, such as a blood clot, formed in a blood vessel and retrieve by suction the object immediately after the cutting, a cutting portion of the object cutting means has a first end connected to a power transmission rod on the leading-end side of the leading-end case portion of the catheter and a second end fixed to an internal wall surface of the leading-end case portion on the base-end side thereof.

### BACKGROUND OF THE INVENTION

The present invention relates to a suction catheter used in a treatment where the suction catheter is inserted into a blood vessel to suck and remove an object such as a blood clot therefrom. Specifically, the present invention relates to a technique used when an object causes an occluded blood-vascular system, such as in the case of cerebral infarction or myocardial infarction, for the purpose of retrieving and removing the object easily and smoothly from the blood vessel without allowing the object to flow downstream.

A number of diseases are known to be caused by an occluded blood vessel. For example, each of the cerebral infarction and the myocardial infarction is a disease caused by an occluded artery system of the corresponding organ. The economy-class syndrome is a potentially fatal disease that is caused by an occluded pulmonary blood vessel due to a blood clot formed in a leg vein.

The blood clot formed in a blood vessel is conventionally removed by use of an apparatus inserted into the blood vessel mainly in accordance with any one of the following two known methods. In one method, an object, such as a blood clot, is removed by being sucked with an opening portion at the leading end of a suction catheter being brought into direct contact with the object. In the other method, the blood clot is firstly fractured, and the fragments of the blood clot are then sucked out by a suction catheter.

For example, the following techniques have been proposed. A suction catheter employed in a proposed technique (see Japanese Patent Application Publication No. 2005-95410) includes: a catheter main body to be inserted into a blood vessel; and a vibrator that make the catheter main body vibrate. The vibrating catheter main body is brought into contact with a blood clot, and thus the blood clot is gradually fractured from the surface by the vibration. According to this technique, the blood clot can be quickly removed without damaging the blood vessel.

A suction catheter employed in another proposed technique (see Japanese Patent Application Publication No. Hei 9-192230) includes an actuator provided near the leading end of the catheter. When the actuator is made to vibrate, the vibration in turn makes the leading end of the catheter vibrate. The leading end of the catheter or an irregular face formed at the leading end is used for chipping away the surroundings.

A suction catheter employed in still another proposed technique (see Japanese Patent Application Publication No. 2006-271693) includes a taper-shaped spiral portion provided at a leading-end portion of the catheter main body. The spiral portion has a coil-shaped piece wound up by at least one turn. A catheter for fracturing objects is disposed inside the spiral portion, and ejects a jet stream of a liquid to fracture an object (for example, a blood clot) in the blood vessel. The object thus fractured is then sucked up through the leading-end portion, and is taken to the outside.

A suction catheter employed in still another proposed technique (see Japanese Patent Application Publication No. 2006-263125) includes a tubular body that has an opening for ejection and an opening for suction. To dissolve an object to be dissolved, a dissolving agent is ejected through the opening for ejection to the object to be dissolved. Parts of the object to be dissolved may remain undissolved. Such fine fragments of the object to be dissolved are sucked into the tubular body through the opening for suction. In addition, mechanical means configured to generate vibrations is provided to the tubular body to fracture the object to be dissolved. With the suction catheter described above, fine fragments of a blood clot that has been fractured and is left undissolved are sucked through the opening for suction.

A suction catheter employed in still another proposed technique (see Japanese Patent Application Publication No. 2000-279525) is provided with a vibrator. An irregular-surface portion is formed in the outer circumferential surface of the leading-end portion of the suction catheter. The vibrator makes the irregular-surface portion vibrate, and the irregular-surface portion chips away the atheroma formed in the arteriosclerotic narrowed lesion. The chipped fragments of the atheroma are sucked and taken out of the body through the opening portion for suction at the leading-end portion of the catheter.

However, either of the means described in Japanese Patent Application Publication No. 2005-95410 and the means described in Japanese Patent Application Publication No. Hei 9-192230 has an insufficient ability to remove a blood clot, and does not allow easy and smooth removal of the blood clot.

### SUMMARY OF THE INVENTION

In each of the means described in Japanese Patent Application Publication No. 2006-271693, Japanese Patent Application Publication No. 2006-263125 and Japanese Patent Application Publication No. 2000-279525, the position where the blood clot is subjected to fracturing or the like is away from the position where the blood clot is retrieved. For this reason, when the direction of the blood flow is not favorable, the fragments of the blood clot may possibly flow downstream. For example, when a blood clot is removed from a blood vessel in the head, the catheter has to be inserted from the upstream side of the blood clot in the blood flow. Accordingly, the fragments of the blood clot readily flow downstream, unless the fragments of the blood clot are retrieved substantially simultaneously with the fracturing or the like. When the original blood clot is formed in an artery system, the fragments of the blood clot that have flowed downstream may cause a new blood clot to be formed in a peripheral capillary.

Accordingly, requirements for a suction catheter used for sucking and removing an object, such as a blood clot, formed in a blood vessel are as follows.

1) To have a sufficient ability to remove the blood clot.

2) To be capable of preventing fragments of the blood clot from flowing downstream.

3) To allow safe treatment.

4) To be capable of following the complex bending of the blood vessel.

However, none of the suction catheters proposed thus far satisfies all the above-mentioned requirements. Accordingly, there has been a demand for a proposal of a suction catheter that can retrieve and remove easily and smoothly an object, such as a blood clot, formed in a blood vessel from the blood vessel without allowing the object to flow downstream.

In this respect, as shown in FIG. 17, the present inventors have already proposed a suction catheter 100 including object cutting means 103 that is provided at a leading-end portion of a catheter main body 101 and that is movable in the axial direction (see Japanese Patent No. 4267055). The suction catheter 100 is configured as follows. Specifically, a base-end portion of the catheter main body 101 is connected to a suction apparatus, and a lumen 111 of the catheter main body 101 is vacuumed, so that the object cutting means 103 is operated to cut an object that sticks to the internal wall of the blood vessel, and to suck and retrieve the object immediately after the cutting.

Specifically, this suction catheter 100 includes: a catheter main body 101 made of a flexible lengthy body having a lumen 111 therein; a leading-end case portion 102 positioned at a leading-end portion of the catheter main body 101 and having an opening portion 104 for retrieving an object; and object cutting means 103 that is partially exposed through the opening portion 104 and is movable in the axial direction within the leading-end case portion 102. In addition, the object cutting means 103 has a cutting portion 131 exposed through the opening portion, and a power receiving portion 132 that moves the cutting portion 131 upon reception of power from the outside.

In the above-described suction catheter 100 proposed by the present inventors, a first end 131a of the cutting portion 131 is fixed to a leading-end inner surface 102a of the leading-end case portion 102, and a second end 131b of the cutting portion 131 is connected to the power receiving portion 132 on a base-end side of the leading-end case portion 102. Hence, the moving distance may be different between the leading-end side and the base-end side depending on the structure of the cutting portion 131 or the nature of the material of the cutting portion 131.

Specifically, as indicated by an arrow in FIG. 18A, when the lumen 111 is vacuumed, the power receiving portion 132 moves toward the base end. With this, the cutting portion 131 moves as indicated by the dotted arrow in the drawing. Here, since the cutting portion 131 is pulled on the second end 131b side, the intervals of cutting regions may be larger on the second end 131b side and smaller on the first end 131a side. When the intervals of the cutting regions in FIG. 18B are denoted by d1, d2, d3, d4, d5, d6, and d7 from the first end 131a side to the second end 131b side, the sizes of the intervals of the cutting regions have the following relationship: d1<d2<d3<d4<d5<d6<d7.

Accordingly, the above-described suction catheter 100 proposed by the present inventors can retrieve and remove easily and smoothly an object, such as a blood clot, formed in a blood vessel from the blood vessel without allowing the object to flow downstream. However, the intervals of the cutting regions may be small on the first end 131a side of the cutting portion 131 depending on the structure of the cutting portion 131 and the nature of the material of the cutting portion 131. Hence, it may be difficult for the suction catheter 100 to effectively cut the object on the leading-end side of the suction catheter 100. In sum, it is desirable that the object cutting operation on the leading-end side be effectively carried out, because what comes into contact with the object in a blood vessel is the leading-end portion of the suction catheter 100.

The present invention has been made in view of the above-described circumstances, and an object of the present invention is to provide a suction catheter that can effectively cut an object, such as a blood clot, formed in a blood vessel on a leading-end side of the suction catheter and that is configured to easily and smoothly retrieve and remove the object from the blood vessel without allowing the object to flow downstream, as well as a suction catheter system that enables efficient operation of the suction catheter.

### Solution to Problem

To achieve the above-described object, a suction catheter of the present invention includes:

a catheter main body made of a flexible lengthy body with a lumen formed therein;

a leading-end case portion positioned at a leading-end portion of the catheter main body and having an opening portion for retrieving an object; and

object cutting means that is partially exposed through the opening portion of the leading-end case portion and is movable in an axial direction within the leading-end case portion,

the suction catheter being adapted to be used in a treatment where a leading-end side of the catheter main body is inserted into a blood vessel together with the leading-end case portion, a base-end portion of the catheter main body is connected to a suction apparatus, and the lumen is vacuumed by means of the suction apparatus, so that the object cutting means is operated to cut an object, which sticks to an internal wall of the blood vessel, at the opening portion of the leading-end case portion, and also to suck and retrieve the object through the opening portion into the catheter main body, wherein

the object cutting means includes
a cutting portion that includes a plurality of lines of cutting regions each extending in a substantially perpendicular direction to the axial direction and that has a resilience to restore changed intervals of adjacent ones of the cutting regions,
a power receiving portion that is movable in the axial direction upon reception of power from the outside, and
a power transmission rod that connects the cutting portion to the power receiving portion and that transmits the power received by the power receiving portion to the cutting portion, and

the cutting portion has a first end connected to the power transmission rod on the leading-end side of the leading-end case portion, and a second end fixed to an internal wall surface of the leading-end case portion on a base-end side thereof.

Moreover, the suction catheter of the present invention is preferably the above-described suction catheter, in which the cutting portion is a spiral body formed by winding up a wire-like member a number of times.

Moreover, the suction catheter of the present invention is preferably the above-described suction catheter, in which the cutting portion is the spiral body formed of the wire-like member, and the wire-like member has irregularities on at least surfaces of the cutting regions.

Moreover, the suction catheter of the present invention is preferably the above-described suction catheter, in which the cutting portion is the spiral body formed of the wire-like member, and the wire-like member has a polygonal cross-sectional shape.

Moreover, the suction catheter of the present invention is preferably the above-described suction catheter, in which the cutting portion is a bellows-shaped body including a sheet-like member or tubular member having mountain (peak) fold portions and valley fold portions repeatedly formed therein.

Moreover, the suction catheter of the present invention is preferably any one of the above-described suction catheters, in which the cutting portion has a width or diameter gradually increasing from the leading-end side to the base-end side.

Moreover, the suction catheter of the present invention is preferably any one of the above-described suction catheters, in which the leading-end case portion includes a locking stopper formed in an internal wall surface of the leading-end case portion, the locking stopper restricting a movement of the object cutting means toward the base-end portion of the catheter main body.

Moreover, the suction catheter of the present invention is preferably any one of the above-described suction catheters, in which at least a part of any one of the leading-end case portion and the object cutting means is made of a metal member.

Moreover, the suction catheter of the present invention is preferably any one of the above-described suction catheters, in which the opening portion is provided in a leading-end inclined face of an obliquely-formed leading-end portion of the leading-end case portion.

Moreover, the suction catheter of the present invention is preferably anyone of the above-described suction catheters, in which the opening portion is provided in a leading-end side circumferential-body surface of the leading-end case portion.

Moreover, the suction catheter of the present invention is preferably any one of the above-described suction catheters, in which the power receiving portion includes a magnetic material, and the leading-end case portion includes magnetic-force generating means provided to the internal wall surface of the leading-end case portion and configured to supply power to the power receiving portion.

Moreover, the suction catheter of the present invention is preferably any one of the above-described suction catheter, in which at least one of the catheter main body and the leading-end case portion includes a guide-wire hole formed in an outer surface thereof, the guide-wire hole allowing a guide-wire to be inserted thereinto.

In the suction catheter of the present invention, the object cutting means that is provided in the leading-end portion of the catheter main body made of the flexible lengthy body with the lumen formed therein and is movable within the leading-end case portion includes the cutting portion in which the intervals of adjacent ones of the cutting regions are variable, the power receiving portion that is movable in the axial direction upon reception of power from the outside, and the power transmission rod that transmits the power received by the power receiving portion to the cutting portion. In addition, the cutting portion has the first end connected to the power transmission rod on the leading-end side of the leading-end case portion and a second end fixed to the internal wall surface of the leading-end case portion on the base-end side thereof.

Hence, the power transmitted from the power receiving portion is received on the first end side of the cutting portion positioned on the leading-end side of the suction catheter, and the object cutting means operates in such a manner that the intervals of the cutting regions can change preferentially on the first end side and more greatly. Thus, it is possible to solve such a problem that the change in the intervals of the cutting regions is reduced on the leading-end side depending on the structure of the cutting portion or the nature of the material of the cutting portion.

Accordingly, it is possible to provide a suction catheter that can effectively cut an object, such as a blood clot, formed in a blood vessel on a leading-end side of the suction catheter, and that is configured to easily and smoothly retrieve and remove the object from the blood vessel without allowing the object to flow downstream, as well as a suction catheter system that enables efficient operation of the suction catheter.

### BRIEF DESCRIPTION OF THE DRAWINGS

FIGS. 1A to 1C are views illustrating a suction catheter according to the present invention. FIG. 1A is a schematic sectional view illustrating the suction catheter before its cutting action. FIG. 1B is a schematic sectional view illustrating the suction catheter after its cutting action. FIG. 1C is an enlarged schematic plan view illustrating the portion (cutting portion) indicated by the sign A and surrounded by the dashed-dotted line in FIG. 1B.

FIGS. 2A to 2C are views illustrating a series of actions of the object cutting means of the suction catheter according to the present invention. FIG. 2A is a schematic sectional view illustrating the state before a sucking force is applied. FIG. 2B is a schematic sectional view illustrating the state where a strong sucking force is applied. FIG. 2C is a schematic sectional view illustrating the state where a weak sucking force is applied.

FIGS. 3A and 3B are views illustrating an example of means configured to make the sucking force stronger or weaker. FIG. 3A is a schematic view illustrating the state of applying a strong sucking force. FIG. 3B is a schematic view illustrating the state where the force is switched to a weak sucking force.

FIGS. 4A to 4C are schematic sectional views illustrating a series of steps of cutting and removing an object (blood clot) by using a suction catheter according to the present invention. FIG. 4A is a schematic sectional view illustrating the state where the suction catheter is inserted into a blood vessel. FIG. 4B is a schematic sectional view illustrating the state where a leading-end face of the suction catheter is brought into contact with the blood clot. FIG. 4C is a schematic sectional view illustrating the state where the blood clot is cut with the object cutting means, and the resultant fragments of the blood clot are retrieved.

FIGS. 5A and 5B are views illustrating a retrieval channel for the object (blood clot) formed in the suction catheter according to the present invention. FIG. 5A is a schematic sectional view illustrating the state where the retrieval channel is fully opened. FIG. 5B is a schematic sectional view illustrating the state where the retrieval channel is partially closed by a power receiving portion of the object cutting means.

FIG. 6 is a schematic sectional view illustrating a suction catheter according to another embodiment of the present invention.

FIG. 7 is a schematic sectional view illustrating a suction catheter according to still another embodiment of the present invention.

FIG. 8 is a schematic sectional view illustrating a suction catheter according to still another embodiment of the present invention.

FIG. 9 is a schematic sectional view illustrating the state where an object (blood clot) is cut and removed by using the suction catheter of the present invention shown in FIG. 8.

FIG. 10 is a schematic sectional view illustrating a suction catheter according to still another embodiment of the present invention.

FIG. 11 is a schematic sectional view illustrating the state where an object (blood clot) is cut and removed by using the suction catheter of the present invention shown in FIG. 10.

FIGS. 12A to 12D are views illustrating examples of cross-sectional shapes of cutting portions used in the present invention. FIG. 12A is an enlarged schematic sectional view of a cutting portion having semi-spherical raised portions in its surface. FIG. 12B is an enlarged schematic sectional view of a cutting portion having pyramidal raised portions in its surface. FIG. 12C is an enlarged schematic sectional view of a cutting portion having semi-spherical recessed portions in its surface. FIG. 12D is an enlarged schematic sectional view of a cutting portion having wavy line-shaped recessed portions in its surface.

FIGS. 13A to 13D are views illustrating examples of cross-sectional shapes of cutting portions used in the present invention. FIG. 13A is an enlarged schematic sectional view of a cutting portion having an up-pointing triangular cross-sectional shape. FIG. 13B is an enlarged schematic sectional view of a cutting portion having a down-pointing triangular cross-sectional shape. FIG. 13C is an enlarged schematic sectional view of a cutting portion having a rhombic (diamond-like) cross-sectional shape. FIG. 13D is an enlarged schematic sectional view of a cutting portion having a regular star polygonal (regular star octagonal) cross-sectional shape.

FIGS. 14A and 14B are views illustrating other examples of cutting portions used in the present invention. FIG. 14A is a schematic view illustrating a bellows-shaped body including a sheet-like member having mountain fold portions and valley fold portions repeatedly formed therein. FIG. 14B is a schematic view illustrating a bellows-shaped body including a sheet-like member having mountain fold portions and valley fold portions repeatedly formed therein, and having a width (diameter) gradually increasing from the leading-end side to the base-end side.

FIG. 15 is a view illustrating another example of a cutting portion used in the present invention, i.e., a schematic view illustrating a bellows-shaped body including a tubular member having mountain fold portions and valley fold portions repeatedly formed therein.

FIGS. 16A to 16C are views illustrating another example of a cutting portion used in the present invention. FIG. 16A is a front view of a leaf spring member to constitute a bellows-shaped body. FIG. 16B is a side view of the leaf spring member. FIG. 16C is a schematic view illustrating a bellows-shaped body formed by connecting leaf spring members that face alternately in different directions.

FIG. 17 is a schematic sectional view illustrating a conventional suction catheter.

FIGS. 18A and 18B are views illustrating the conventional suction catheter. FIG. 18A is a view illustrating the state where a cutting portion moves toward the base-end. FIG. 18B is an enlarged schematic plan view illustrating the cutting portion in FIG. 18 A.

### DETAILED DESCRIPTION OF THE INVENTION

Hereinafter, some exemplary embodiments of the present invention will be described with reference to the drawings.

Note that the embodiments described below are preferred specific examples of the present invention, and hence have various technical limitations. However, the scope of the present invention is not limited to these embodiments, unless a specific remark stating that the present invention is limited is given in the following description.

A suction catheter to be provided according to the present invention is used in a treatment where a leading-end side of the suction catheter is inserted into a blood vessel, while the base-end portion is connected to a suction apparatus, and the lumen is vacuumed with this suction apparatus, so that an object in the blood vessel is sucked and retrieved.

To put it differently, the suction catheter of the present invention is used together with suction means (a suction apparatus). When an object causes an occluded blood-vascular system, such as in the case of cerebral infarction or myocardial infarction, the suction catheter is inserted deep into the blood vessel until the suction catheter can reach the lesion. The object such as a blood clot (hereafter, simply referred to as a “blood clot”), which causes the occlusion of the blood vessel, is sucked directly by the suction catheter, and is thus removed out of the body. Note that the suction apparatus is an ordinary apparatus that is commonly used in the above-mentioned treatment. Accordingly, no description of the suction apparatus will be given in the following embodiments.

### First Embodiment

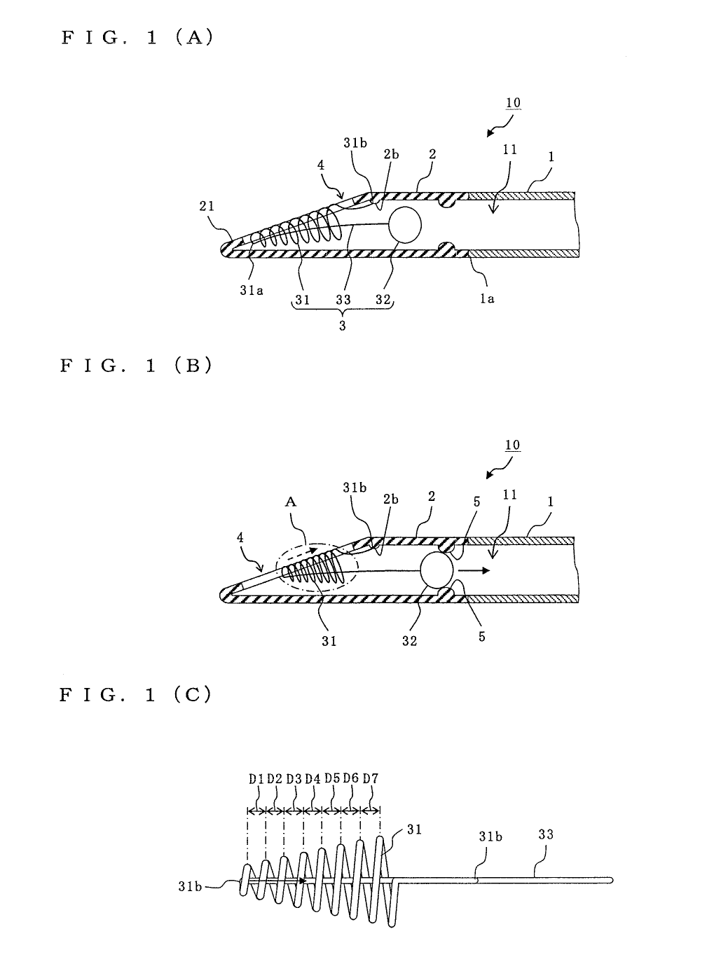

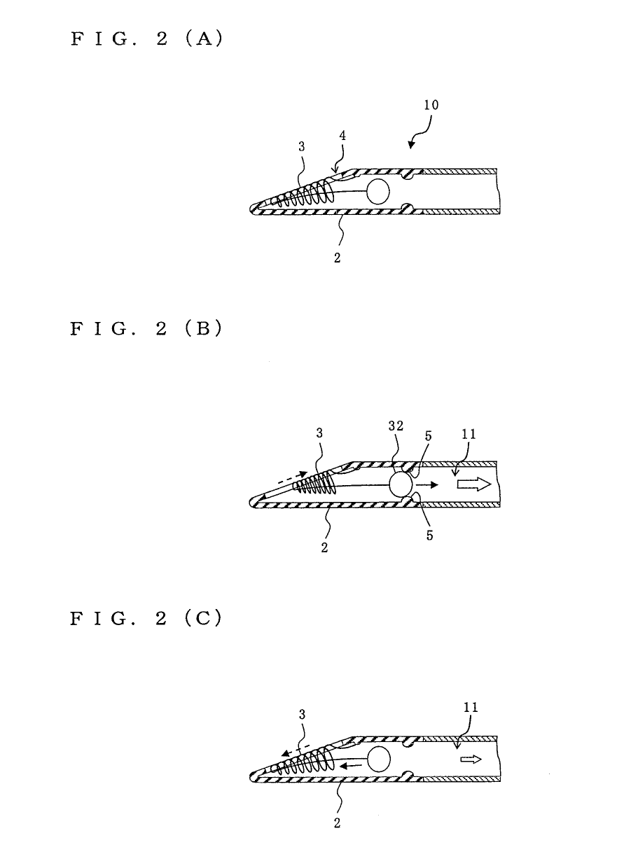

As shown in FIG. 1, a suction catheter 10 according to this embodiment includes: a catheter main body 1; a leading-end case port ion 2 positioned at a leading-end portion 1a of the catheter main body 1; and object cutting means 3 provided in the leading-end case portion 2.

The catheter main body 1 is made of a flexible lengthy body with a lumen 11 formed therein. Hence, the catheter main body 1 is capable of sufficiently following the bending of the blood vessel.

The catheter main body 1 can be made of various materials. Some examples of such materials are polyvinyl chloride; polyethylene; polypropylene; polyurethane; ethylene-propylene copolymer; ethylene-vinyl acetate copolymer; other polyolefins; polyethylene terephthalate; polybutylene terephthalate; other polyesters; polyamide; polyimide; polytetrafluoroethylene; polyvinylidene fluoride; other fluorinated resins; various other thermoplastic resins; various other thermosetting resins; polyamide elastomer; polyester elastomer; other thermoplastic elastomers; and various rubbers.

The outer diameter of the catheter main body 1 is not particularly limited, and is preferably made as small as possible in order to allow removal of a blood clot in a capillary. A preferable outer diameter is, for example, approximately 1 mm.

The leading-end case portion 2 has an opening portion 4 through which a blood clot is retrieved. The opening portion 4 also serves as an inlet through which the blood clot is sucked into the lumen 11 of the vacuumed catheter main body 1. Accordingly, the opening portion 4 is provided to face a blood clot that sticks to an internal wall of a blood vessel.

As shown in FIGS. 1A and 1B, the leading-end portion of the leading-end case portion 2 is formed obliquely to be a leading-end inclined face 21, and the opening portion 4 is provided in a part of the leading-end inclined face 21.

The leading-end case portion 2 may be formed integrally with or separately from the catheter main body 1.

The object cutting means 3 is movable in the axial direction within the leading-end case portion 2. Here, the axial direction is the same direction as the direction of the longitudinal axis of the catheter main body 1.

The object cutting means 3 includes a cutting portion 31 partially exposed as cutting regions through the opening portion 4; a power receiving portion 32 that is movable in the axial direction upon reception of power from the outside; and a power transmission rod 33 that connects the cutting portion 31 to the power receiving portion 32 and that transmits power received by the power receiving portion 32 to the cutting portion 31.

The cutting portion 31 includes cutting regions provided to traverse in a substantially perpendicular direction to the axial direction in which the object cutting means 3 is movable. The substantially perpendicular direction to the direction in which the object cutting means 3 is movable here refers to the direction in which the cutting regions draw plane trajectories, when moves along with the motion of the object cutting means 3. Meanwhile, the cutting regions refer to portions to be brought into contact with an object.

In addition, the cutting portion 31 is formed to have a retrieving opening for immediately taking the blood clot, which has been cut, into the leading-end case portion 2. Some examples of the cutting portion 31 formed to have the retrieving opening are an arch-shaped body formed of a wire-like member with only one cutting region formed and a spiral body formed by winding up a wire-like member a number of times with gaps formed between two adjacent lines to form multiple cutting regions.

When the cutting portion 31 is made of an arch-shaped body as described above, the shape of the wire-like member portion provided to traverse in the substantially perpendicular direction to the axial direction in which the object cutting means 3 is movable can be an arc shape that is similar to the shape of an internal wall of a blood vessel. Hence, the cutting portion 31 made of the arch-shaped body can easily achieve efficient removal of the blood clot.

In addition, the cutting portion 31 is preferably made of a spiral body including multiple lines of cutting regions arranged in the axial direction and connected in series. The cutting portion 31 has a resilience to restore changed (decreased) intervals of adjacent ones of the cutting regions (to restore the original state). When the cutting portion 31 is made of a spiral body having multiple cutting regions arranged into multiple lines, the cutting portion 31 secures the spring resilience, and also parts of the wire-like member serve as the cutting regions, so that multiple objects that stick to an internal wall of a blood vessel can be cut efficiently and simultaneously.

In FIGS. 1A to 1C, the cutting portion 31 is illustrated as a spiral body (coil-spring shape). The cutting portion 31 has a first end 31a connected to the power transmission rod 33 on the leading-end side of the leading-end case portion 2, and the cutting portion 31 is thus connected to the power receiving portion 32 through the power transmission rod 33.

Meanwhile, a second end 31b of the cutting portion 31 is fixed to an internal wall surface 2b on the base-end side of a leading-end case portion 4. In other words, the second end 31b of the cutting portion 31 is fixed to the internal wall surface 2b that is closer to the base-end than the position at which the first end 31a of the cutting portion 31 and the power transmission rod 33 are connected to each other is, and than the opening portion 4 of the leading-end case portion 2 is.

Accordingly, the use of the suction catheter 10 of the present invention requires driving means configured to supply power to the power receiving portion 32 of the object cutting means 3, and the suction catheter 10 and the driving means (not illustrated) constitute a suction catheter system in the present invention.

An example of an external power to move the power receiving portion 32 is the sucking force to attract the power receiving portion 32. The use of the sucking force as the power for moving the object cutting means 3 allows a suction apparatus (not illustrated) used for vacuuming the lumen 11 of the catheter main body 1 and for sucking and retrieving a blood clot to be also used as the source of the sucking force.

In this case, no additional driving means configured to supply the power needs to be provided, so that the fracturing of and the retrieving of the blood clot can be carried out economically and with efficiency both in design and in operation.

In addition, the use of the sucking force as the power to attract the power receiving portion 32 allows the power receiving portion 32 to be formed as, for example, a spherical voluminous portion attached to the first end 31a of the spiral body cutting portion 31 through the power transmission rod 33.

The power transmission rod 33 transmits power received by the power receiving portion 32 to the cutting portion 31, and causes the cutting portion 31 to extend and contract in the axial direction, along with the motion of the power receiving portion 32, so that the intervals of the cutting regions can change.

Now, suppose a case where a suction apparatus (not illustrated) is used for vacuuming the lumen 11 of the catheter main body 1 of the suction catheter 10 of this embodiment shown in FIG. 1A. In such a case, a sucking force is produced as indicated by the outline arrow in FIG. 1B. Accordingly, a power is supplied to the power receiving portion 32, and the power receiving portion 32 moves in the direction indicated by the solid arrow. In addition, along with the motion of the power receiving portion 32, the cutting portion 31 contracts in the direction indicated by the dotted arrow to make narrower the intervals in the wire-like member constituting the cutting portion 31.

Here, the cutting portion 31 is pulled toward the base-end by the first end 31a. However, since the second end 31b is fixed, the base-end of the cutting regions does not retract greatly. Hence, the intervals of the cutting regions of the cutting portion 31 change preferentially from the first end 31a side, and the change becomes great. Here, since the power is transmitted uniformly to the cutting portion 31, the change in intervals of the cutting regions is substantially uniform over the entire cutting regions.

When the intervals of the cutting regions in FIG. 1C are denoted by D1, D2, D3, D4, D5, D6, and D7 from the first end 31a side to the second end 31b side, the intervals of the cutting regions are such that D1=D2=D3=D4=D5=D6=D7.

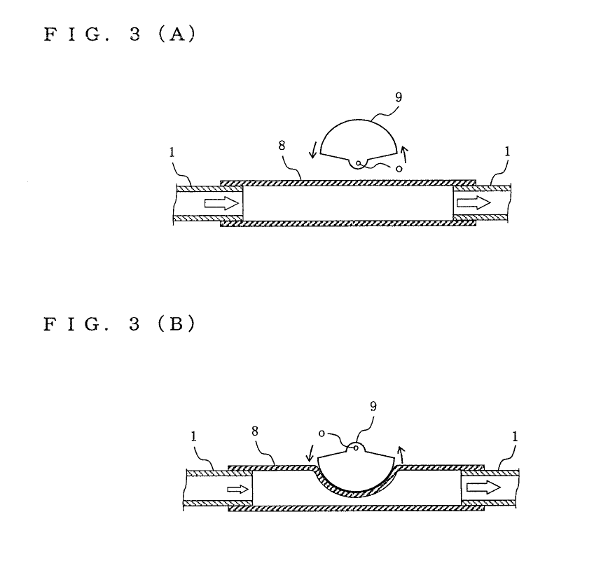

In addition, when the cutting portion 31 is formed as a spiral body in this embodiment, the spring resilience of the cutting portion 31 allows the object cutting means 3 to move back and forth as shown in FIGS. 2A to 2C. FIGS. 2A to 2C are views illustrating a series of actions of the object cutting means 3 in the suction catheter 10.

The back and forth motion of the object cutting means 3 can be achieved by adjusting the strength of the power in the driving means configured to supply power to the power receiving portion 32. In this embodiment, the strength of the power is adjusted by making the sucking pressure stronger or weaker.

Specifically, while the driving means (not illustrated) is not in operation, the suction catheter 10 stays in a state where the object cutting means 3 (the cutting portion 31) is extended toward the leading-end portion of the leading-end case portion 2, as shown in FIG. 2A.

Meanwhile, when the lumen 11 is vacuumed by the driving means to supply a strong sucking force to the power receiving portion 32, the power receiving portion 32 is pulled toward the base-end portion of the catheter main body 1 as shown in FIG. 2B. This causes the cutting portion 31 to contract against the spring biasing force (resilience).

Then, when the driving means stops its operation or when the sucking force of the driving means is weakened, the power receiving portion 32 returns to the state shown in FIG. 2C by the spring biasing force of the cutting portion 31 in such a manner that the power receiving portion 32 is released toward the leading-end portion of the leading-end case portion 2.

Note that the spring resilience of the cutting portion 31 formed as the spiral body enables the object cutting means 3 to move back and forth in this embodiment. If the object cutting means 3 does not have an extending and contracting function, a separate extending and contracting portion has to be provided.

In addition, the leading-end case portion 2 in this embodiment includes locking stoppers 5 and 5 on the internal wall surface of the leading-end case portion 2 as shown in FIGS. 1A to 1C and 2A to 2C. The locking stoppers 5 and 5 are designed to restrict the movement of the power receiving portion 32 toward the base-end portion of the catheter main body 1. The locking stoppers 5 and 5 prevent the power receiving portion 32 from being pulled more than necessary, and enable the object cutting means 3 to move back and forth efficiently and smoothly.

Note that the locking stoppers 5 and 5 may be formed integrally with the leading-end case portion 2, or may be formed separately from the leading-end case portion 2 and attached thereto.

Here, an example of the means configured to make the sucking pressure stronger or weaker is means configured to change the size (diameter) of the lumen of the catheter main body 1. Specifically, the sucking force can be made weaker by pressing a middle portion of the catheter main body 1 to reduce the diameter of the lumen, which serves as the suction route. On the other hand, the sucking force can be made stronger by stopping the pressing to increase the diameter of the lumen (to make the diameter of the lumen back to the original size).

Specifically, for example, a lumen diameter variable region that is deformable by pressing is formed in a middle portion of the catheter main body 1 between the object cutting means and the suction apparatus. In addition, a member that moderately presses the lumen diameter variable region is formed.

In FIGS. 3A and 3B, the lumen diameter variable region is shown as an elastic tube 8, such as a rubber tube, provided in a middle portion of the catheter main body 1 between the object cutting means 3 and the suction apparatus, and the pressing member is shown as a depressor 9 that has a substantially sector shape in a side view and that eccentrically rotates about the rotation axis O to moderately press the elastic tube 8 from the outside. Note that the depressor 9 may be, for example, one which is rotated with a motor automatically and continuously at high speed.

Specifically, FIG. 3A shows the state where the elastic tube 8 is not pressed by the depressor 9, and a strong sucking force is being supplied to the object cutting means, and FIG. 3B shows the state where the elastic tube 8 is pressed by the depressor 9 to weaken the sucking force being applied to the object cutting means.

Thus, in the suction catheter 10 of the present invention, the power transmitted from the power receiving portion 32 through the power transmission rod 33 is received on the first end 31a side of the cutting portion 31, and the object cutting means 3 operates, so that the intervals of the cutting regions can be reduced efficiently and preferentially from the first end 31a side. Thus, an object that sticks to an internal wall of a blood vessel can be cut, and also the object can be sucked and retrieved into the catheter main body 1 through the opening portion 4. In addition, by making the sucking force of the suction apparatus stronger and weaker, the cutting portion 31 reciprocally moves in the axial direction, so that an object can be cut continuously and efficiently.

Accordingly, the suction catheter 10 of the present invention is effective, especially when the change in the intervals of the cutting regions is different between the first end 31a side and the second end 31b side because of the material, shape, or the like of the cutting portion 31, i.e., when the change in intervals of the cutting regions is non-uniform between the first end 31a side and the second end 31b side.

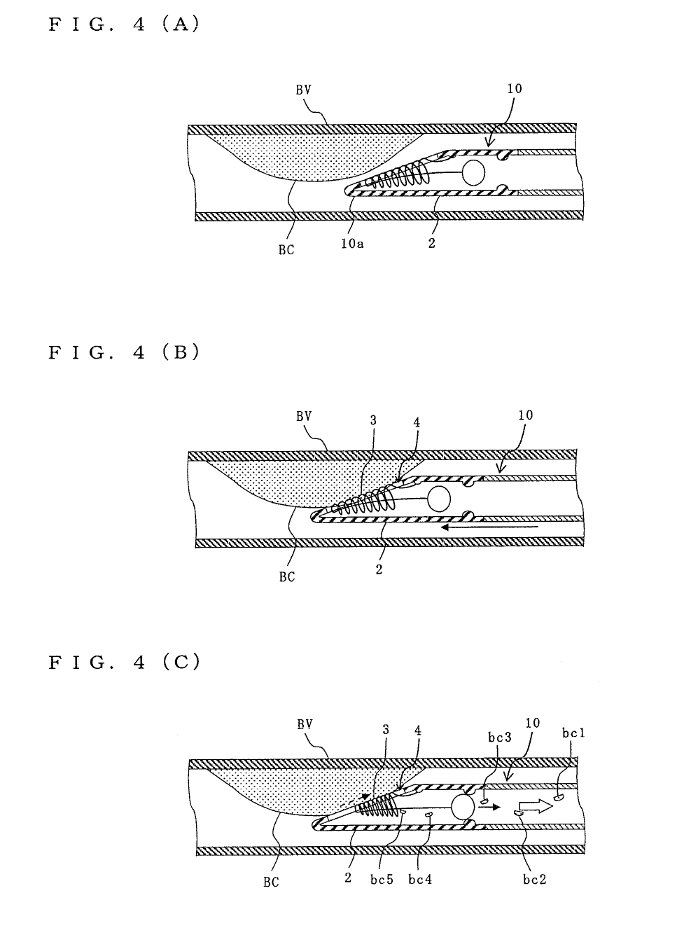

Next, a case where a blood clot in a blood vessel is cut and removed by using the thus configured suction catheter 10 is described. FIGS. 4A to 4C are schematic sectional views illustrating a series of steps of cutting and removing a blood clot by using the suction catheter.

First, as shown in FIG. 4A, the suction catheter 10 is inserted into a blood vessel BV, until a leading-end side 10a of the suction catheter 10 reaches a blood clot BC. Here, the base-end portion of the suction catheter 10 is connected to a suction apparatus.

Then, as shown in FIG. 4B, the cutting portion 31 of the object cutting means 3 exposed through the opening portion 4 of the leading-end case portion 2 is brought into contact with the blood clot BC.

Subsequently, as shown in FIG. 4C, the lumen 11 is vacuumed with this suction apparatus to move the object cutting means 3. The blood clot in the blood vessel is thus cut with the cutting portion 31, and the fragments of the blood clot are securely caught by suction and retrieved.

FIG. 4C shows how to suck and retrieve five blood clot fragments bc1, bc2, bc3, bc4, and bc5 that have been cut from the blood clot BC.

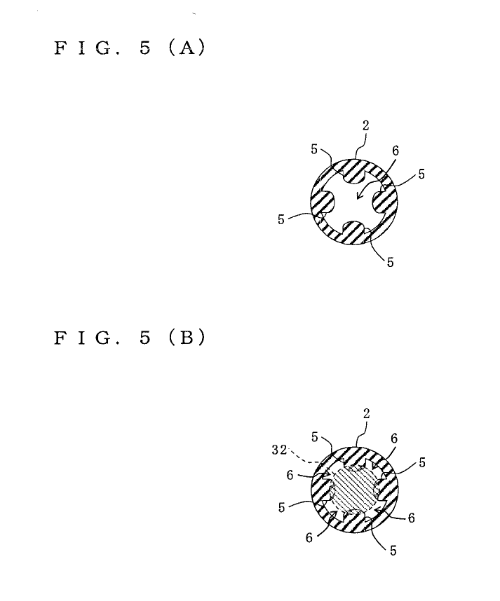

In addition, as shown in FIG. 5A, the locking stoppers 5 and 5 are provided to protrude from some parts of the internal wall surface of the leading-end case portion 2. The rest of the leading-end case portion 2 serves as a retrieval channel 6 used when the fragments of the blood clot are sucked and retrieved. Here, suppose a case where a single locking stopper 5 is provided with a uniform height (thickness) in the internal wall surface of the leading-end case portion 2. In this case, when the power receiving portion 32 pulled toward the base-end portion of the catheter main body 1 is brought into contact with the locking stoppers 5, the power receiving portion 32 may possibly close the retrieval channel 6.

In this respect, the multiple locking stoppers 5 are formed to protrude from some parts of the internal wall surface of the leading-end case portion 2. Accordingly, as shown in FIG. 5B, even when the power receiving portion 32 pulled toward the base-end portion of the catheter main body 1 is brought into contact with the locking stoppers 5, the retrieval channel 6 is not closed completely, and part of the retrieval channel 6 corresponding to the outside of the power receiving portion 32 is left unclosed. For this reason, the fragments of the blood clot are securely sucked and retrieved through the retrieval channel 6.

In addition, it is preferable in this embodiment that at least part of the leading-end case portion 2 or at least part of the object cutting means 3 be made of a metal member. This makes the position of the leading end of the catheter main body 1 observable by means of an X-ray image.

As described above, in this embodiment, the blood clot BC is cut with the cutting portion 31 of the object cutting means 3 exposed through the opening portion 4 provided in the leading-end case portion 2. With this configuration, when the opening portion 4 is brought into contact with the blood clot BC at the beginning of the cutting operation, the blood clot BC is pulled into the leading-end case portion 2 through the opening portion 4 by the negative pressure generated in the lumen 11. In addition, the power receiving portion 32 is also pulled toward the base-end portion of the catheter main body 1 by the negative pressure generated in the lumen 11, so that the cutting portion 31 is stretched out. Then, the cutting portion 31 thus stretched out cuts the blood clot BC, and the fragments of the cut blood clot are immediately sucked into the leading-end case portion 2, by the negative pressure generated in the lumen 11, through the opening portion 4 and the retrieving opening that is a gap formed in the cutting portion 31.

Accordingly, the blood clot can be retrieved and removed easily and smoothly from the blood vessel, while all the fragments of the cut blood are sucked into the leading-end case portion 2, without allowing any of the fragments to flow away. In addition, the cutting portion 31 of the object cutting means 3 exposed through the opening portion 4 cuts only a part of the blood clot BC corresponding to the cutting portion 31. Accordingly, the cutting and the suction of the blood clot BC can be carried out safely without damaging the other parts, such as the internal wall of the blood vessel BV.

In addition, in this embodiment, the object cutting means 3 (the cutting portion 31) is moved back and forth by adjusting the sucking force of the driving means. Thus, when a strong negative pressure generated in the lumen 11 is made weaker, the restoring force of the spring resilience of the cutting portion 31 contracts the cutting portion 31 back to its original length. In addition, while the cutting portion 31 is restoring the original length, the cutting portion 31 cuts the blood clot BC. The fragments of the blood clot thus cut are immediately sucked, by the negative pressure generated in the lumen 11, into the leading-end case portion 2 through the opening portion 4 and the retrieving opening that is a gap formed in the cutting portion 31.

Accordingly, the blood clot BC can be cut and removed efficiently by the repeating of the back-and-forth motion including the action of stretching out the cutting portion 31 and the action of contracting the cutting portion 31 until the restoration of its original length. In addition, the opening portion 4 is provided in the leading-end inclined face 21, which is the obliquely-formed leading-end portion of the leading-end case portion. Accordingly, even when the suction catheter cannot be inserted with the circumferential body portion facing the blood clot, the cutting and removal of the blood clot can be carried out, little by little, starting from the edge portion of the blood clot.

### Second Embodiment

The suction catheter of the present invention can be modified for the purpose of allowing the suction catheter to be efficiently inserted into a blood vessel and of allowing the suction catheter to easily reach a lesion (blood clot). Specifically, a second embodiment is different from the above-described first embodiment in that the suction catheter of the second embodiment includes means configured to assist the insertion of the suction catheter.

Note that, in each of the following other embodiments, points that differ from those of the above-described first embodiment are mainly described. Hence, constituent parts that are the same as those in the first embodiment are denoted by the same reference numerals, and no description will be given for such constituent parts. To put it differently, constituent parts are the same as those of the first embodiment, unless specifically described.

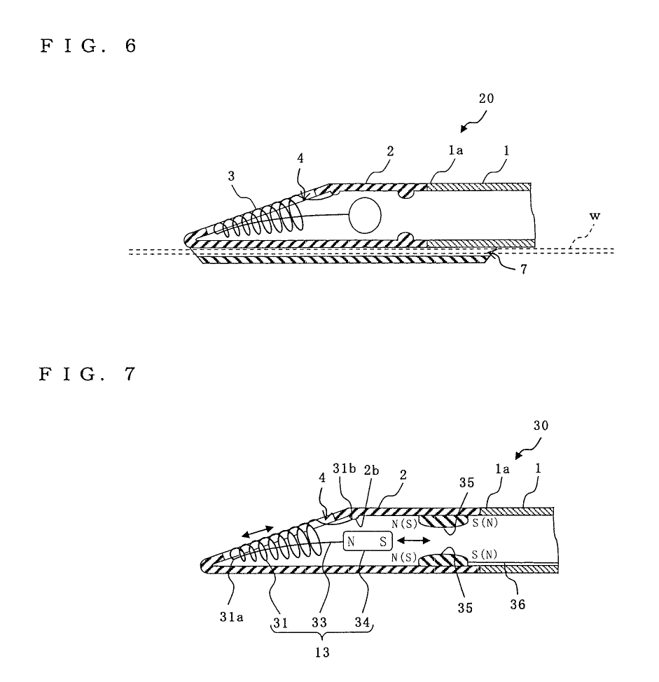

As shown in FIG. 6, a suction catheter 20 of this embodiment includes a catheter main body 1; a leading-end case portion 2 positioned at a leading-end portion 1a of the catheter main body 1, and object cutting means 3 provided in the leading-end case portion 2. In addition, the suction catheter 20 includes a guide-wire hole 7 provided in the outer surface of at least one of the catheter main body 1 and the leading-end case portion 2. A guide-wire W is inserted into the guide-wire hole 7. In other words, the suction catheter 20 includes the guide-wire hole 7 that serves as means configured to assist the insertion of the suction catheter and that is formed in the outer surface in the longitudinal axis direction.

As described above, in this embodiment, the guide-wire W inserted into a blood vessel beforehand is inserted into the guide-wire hole 7, and the suction catheter 20 is inserted along the guide-wire W. The guide-wire hole 7 is thus used as the means configured to assist the insertion of the suction catheter. Accordingly, the suction catheter 20 can be efficiently inserted into the blood vessel.

### Third Embodiment

The suction catheter of the present invention can also be modified for the purpose of allowing the driving means to give the power that is not the sucking force to the object cutting means. Specifically, a third embodiment differs from the above-described first embodiment in that the suction catheter of this third embodiment includes driving means that is not a suction apparatus.

As shown in FIG. 7, a suction catheter 30 of this embodiment includes: a catheter main body 1; a leading-end case portion 2 positioned at a leading-end portion 1a of the catheter main body 1; and object cutting means 13 provided in the leading-end case portion 2.

The object cutting means 13 is made, partially or entirely, of a magnetic material, and includes: for example, a cutting portion 31 exposed through the opening portion 4; a power receiving portion 34 that moves the cutting portion 31 upon reception of a magnetic force from the outside; and a power transmission rod 33 that connects the cutting portion 31 to the power receiving portion 34 and that transmits the power received by the power receiving portion 34 to the cutting portion 31.

In FIG. 7, the cutting portion 31 is shown as a spiral body (coil-spring shape). The cutting portion 31 has a first end 31a connected to the power transmission rod 33 on the leading-end side of the leading-end case portion 2. The cutting portion 31 is connected to the power receiving portion 34 through the power transmission rod 33. Meanwhile, in FIG. 7, a second end 31b of the cutting portion 31 is fixed to the internal wall surface 2b of the leading-end case portion 4 on the base-end side thereof.

The power receiving portion 34 may be made of, for example, a permanent magnet. The permanent magnet may be a ferrite magnet or a rare-earth magnet, and may be of any kind.

The driving means in this embodiment is a magnetic-force apparatus that is capable of attracting or repelling the power receiving portion 34 by means of the magnetic force.

An example of the magnetic-force apparatus is an apparatus including an electromagnet 35 provided to the internal wall surface of the leading-end case portion 2 and a power-supply source that supplies electric power to the electromagnet 35 via an electric wire 36. The electromagnet 35 is capable of generating a magnetic force temporarily when energized. The electromagnet 35 is also capable of reversing the magnetic polarity when the direction of the electric current is reversed.

As described above, in this embodiment, a power can be supplied to the object cutting means 13 by the attraction of the power receiving portion 34 due to the magnetic force (attractive magnetic field) generated by energizing the electromagnet 35 and the attraction of the power receiving portion 34 toward the leading-end inner surface 2a of the leading-end case portion 2 occurring on the basis of the spring biasing force of the cutting portion 31 when the electromagnet 35 ceases to be energized and the magnetic force disappears. Alternatively, a power can be provided to the object cutting means 13 by the attraction (attractive magnetic field) or repulsion (repulsive magnetic field) of the power receiving portion 34 due to the polarity inversion caused by changing the direction of the electric current for energizing the electromagnet 35.

Accordingly, the object cutting means can be moved back and forth without weakening the sucking force for retrieving the fragments of the blood clot.

### Fourth Embodiment

The suction catheter of the present invention can also be modified such that the opening portion that is to be brought into contact with a blood clot faces a different direction. In other words, a fourth embodiment differs from the above-described first embodiment in that the opening portion of this fourth embodiment is provided at a position different from that of the first embodiment.

As shown in FIG. 8, a suction catheter 40 of this embodiment includes: a catheter main body 1; a leading-end case portion 12 positioned at a leading-end portion 1a of the catheter main body 1; and object cutting means 3 provided in the leading-end case portion 12.

The leading-end case portion 12 has multiple opening portions 14 and 14 for retrieving a blood clot. The opening portions 14 also serve as inlets through which a blood clot is sucked into the vacuumed lumen 11 of the catheter main body 1. Accordingly, each of the opening portions 14 is provided to face a blood clot that sticks to an internal wall of a blood vessel.

FIG. 8 shows that the opening portions 14 are provided in a leading-end conical surface 12A of the leading-end case portion 12, and that the leading-end conical surface 12A has a diameter gradually reduced toward the leading end. Note that the conical surface herein may be a surface having a diameter uniformly reduced toward the leading end of the leading-end case portion 12, or may be an arc-shaped surface having a diameter reduced with a degree of the reduction increasing toward the leading end of the leading-end case portion 12.

The leading-end case portion 2 may be formed integrally with or separately from the catheter main body 1.

Parts of the object cutting means 3 are exposed through the opening portions 14 and 14, respectively. In addition, the object cutting means 3 is movable in the axial direction within the leading-end case portion 12.

The object cutting means 3 includes, for example, a cutting portion 31 exposed through the opening portions 14; a power receiving portion 32 that moves the cutting portion 31 upon reception of power from the outside; and a power transmission rod 33 that connects the cutting portion 31 to the power receiving portion 32 and that transmits power received by the power receiving portion 32 to the cutting portion 31.

In FIG. 8, the cutting portion 31 is shown as a spiral body (coil-spring shape). The cutting portion 31 has a first end 31a connected to the power transmission rod 33 on the leading-end side of the leading-end case portion 12. The cutting portion 31 is connected to the power receiving portion 32 through the power transmission rod 33. Meanwhile, in FIG. 8, a second end 31b of the cutting portion 31 is fixed to an internal wall surface 12b of the leading-end case portion 14 on the base-end side thereof.

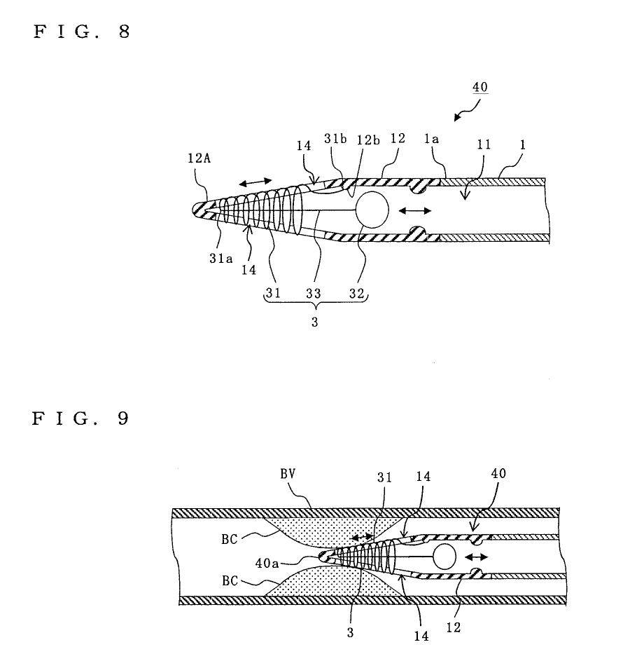

Next, a case where a blood clot in a blood vessel is cut and removed by using the thus configured suction catheter 40 is described. FIG. 9 is a schematic sectional view illustrating how to cut and remove the blood clot by using the suction catheter.

First, as shown in FIG. 9, the suction catheter 40 is inserted into a blood vessel BV, until a leading-end side 40a of the suction catheter 40 reaches the blood clot BC. Then, the cutting portion 31 of the object cutting means 3 exposed through the opening portions 14 of the leading-end case portion 12 is brought into contact with the blood clot BC. Here, the base-end portion of the suction catheter 40 is connected to a suction apparatus. Subsequently, the lumen 11 is vacuumed with the suction apparatus, and the object cutting means 3 is made to move back and forth. The blood clot in the blood vessel can thus be cut by the cutting portion 31, and the fragments of the blood clot can be sucked and retrieved.

As described above, in this embodiment, the multiple opening portions 14 and 14 are provided in the conical surface 12A of the leading-end case portion 12 formed to have a diameter gradually reduced toward the leading end. Accordingly, the suction catheter can be inserted to face a blood clot that is formed in and sticks to a blood vessel in every direction. Hence, a blood clot that is formed in and sticks to a blood vessel in every direction can be cut and removed without rotating the suction catheter in the blood vessel. Accordingly, the use of the suction catheter 40 eliminates the possibility of damaging the inside of the blood vessel by such rotation.

### Fifth Embodiment

The suction catheter of the present invention can also be modified such that the opening portion for contact with a blood clot faces a still different direction. Specifically, a fifth embodiment differs from each of the above-described first and fourth embodiments in that the opening portion of the fifth embodiment is provided at a different position.

As shown in FIG. 10, the suction catheter 50 of this embodiment includes: a catheter main body 1; a leading-end case portion 22 positioned at a leading-end portion 1a of the catheter main body 1; and object cutting means 3 provided in the leading-end case portion 22.

The leading-end case portion 22 has an opening portion 24 through which a blood clot is retrieved. This opening portion 24 also serves as an inlet through which a blood clot is sucked into the vacuumed lumen 11 of the catheter main body 1. Accordingly, the opening portion 24 is provided to face a blood clot that sticks to an internal wall of a blood vessel.

FIG. 10 shows that the opening portion 24 is provided in a leading-end side circumferential-body surface 22A of the leading-end case portion 22 having a cylindrical shape.

This leading-end case portion 22 may be formed integrally with or separately from the catheter main body 1.

The object cutting means 3 is partially exposed through the opening portion 24, and is movable in the axial direction within the leading-end case portion 22.

This object cutting means 3 includes: for example, a cutting portion 31 exposed through the opening portion 24; a power receiving portion 32 that moves the cutting portion 31 upon reception of power from the outside; and a power transmission rod 33 that connects the cutting portion 31 to the power receiving portion 32 and that transmits power received by the power receiving portion 32 to the cutting portion 31.

In FIG. 10, the cutting portion 31 is shown as a spiral body (coil-spring shape). The cutting portion 31 has a first end 31a connected to the power transmission rod 33 on the leading-end side of the leading-end case portion 22. The cutting portion 31 is connected to the power receiving portion 32 through the power transmission rod 33. On the other hand, a second end 31b of the cutting portion 31 is fixed to an internal wall surface 22b of the leading-end case portion 22 on the base-end side thereof.

Next, a case where a blood clot in a blood vessel is cut and removed by using the thus configured suction catheter 50 is described. FIG. 11 is a schematic sectional view illustrating how to cut and remove a blood clot by using the suction catheter.

First, as shown in FIG. 11, the suction catheter 50 is inserted into a blood vessel BV, until a leading-end side 50a of the suction catheter 50 reaches a blood clot BC. Then, the cutting portion 31 of the object cutting means 3 exposed through the opening portion 24 of the leading-end case portion 22 is brought into contact with the blood clot BC. Here, the base-end portion of the suction catheter 50 is connected to a suction apparatus. Subsequently, the lumen 11 is vacuumed with the suction apparatus, and the object cutting means 3 is moved back and forth. The blood clot in the blood vessel can thus be cut by the cutting portion 31, and the fragments of the blood clot can be sucked and retrieved.

As described above, in this embodiment, the opening portion 24 is provided in the leading-end side circumferential-body surface 51 of the leading-end case portion 22 having a cylindrical shape. Hence, the suction catheter can be inserted substantially in parallel with the wall surface of the blood vessel. Accordingly, the blood clot can be cut and removed in the direction from the inside of the blood vessel to the wall surface.

### Sixth Embodiment

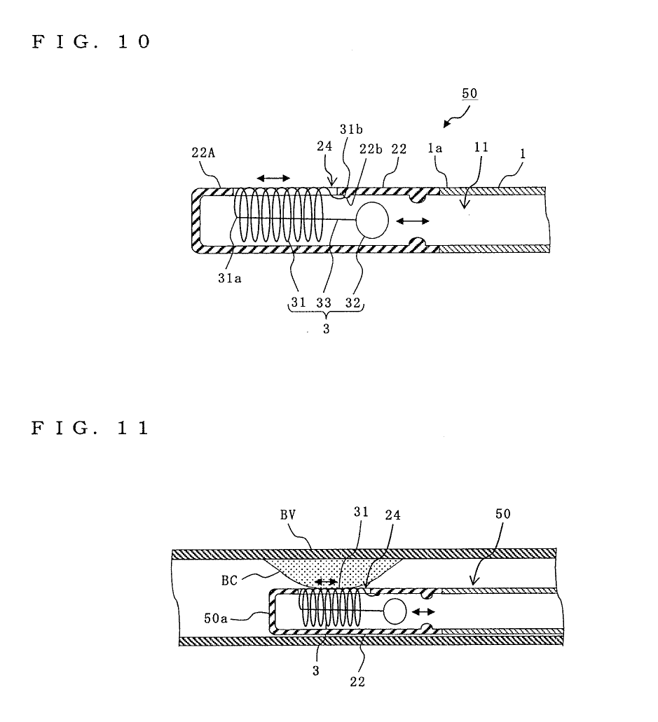

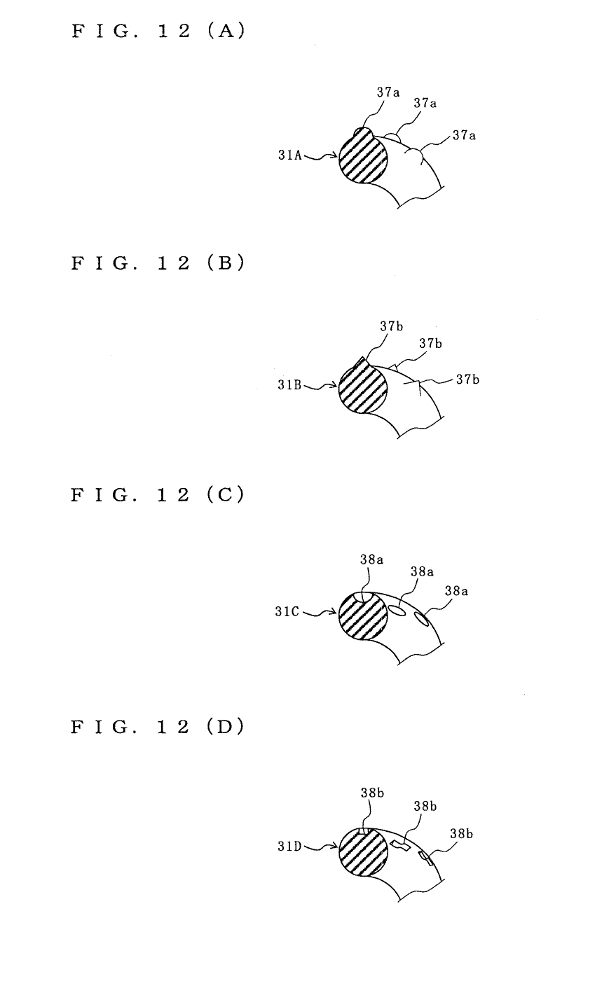

The suction catheter of the present invention can also be modified such that the cutting portion 31 made of the spiral body (coil-spring shape) of any one of the above-described first to fifth embodiments is made of a wire-like member having uneven surfaces at least in the cutting regions. For example, as shown in FIG. 12, it is preferable that the surfaces be made irregular by forming raised portions having a semi-spherical shape, a pyramidal shape, or the like on the surface of the cutting portion made of the wire-like member or by forming semi-spherical dents or line-shaped recessed portions (groove portions) having a line-like shape.

Specifically, FIG. 12A shows a cutting portion 31A made of a wire-like member having multiple semi-spherical raised portions 37a on its surface. FIG. 12B shows a cutting portion 31B made of a wire-like member having multiple pyramidal raised portions 37b on its surface. FIG. 12C shows a cutting portion 31C made of a wire-like member having multiple semi-spherical recessed portions 38a on its surface. FIG. 12D show a cutting portion 31D made of a wire-like member having multiple wavy line-shaped recessed portions 38b on its surface.

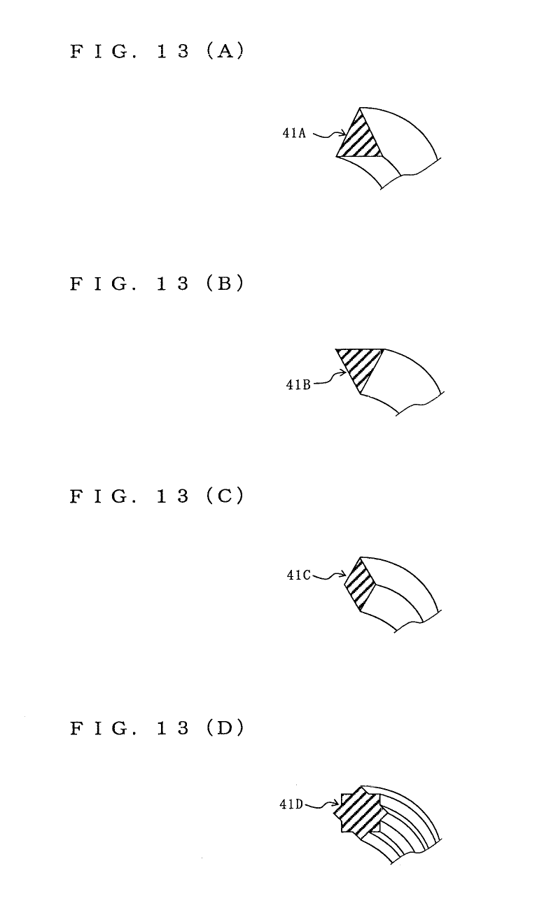

Alternatively, to make the surface of the wire-like member uneven, the cross section of the cutting portion made of the wire-like member may have, for example, any one of polygonal shapes shown in FIGS. 13A to 13D.

Specifically, FIG. 13A shows a cutting portion 41A made of a wire-like member having an up-pointing triangular cross-sectional shape. FIG. 13B shows a cutting portion 41B made of a wire-like member having a down-pointing triangular cross-sectional shape. FIG. 13C shows a cutting portion 41C made of a wire-like member having a rhombic (diamond-like) cross-sectional shape. FIG. 13D shows a cutting portion 41D made of a wire-like member having a regular star polygonal (regular star octagonal) cross-sectional shape.

As described above, the cutting portion is made of the wire-like member having raised portions or recessed portions formed in the surfaces of the cutting regions. Hence, an object is caught by the raised portions or the recessed portions, so that the object can be cut effectively. Moreover, when the cutting portion is made of a wire-like member having a polygonal cross-sectional shape in the cutting regions, the object is caught by corner portions, so that the object can be cut effectively.

Note that, although illustration is omitted, the surface of the wire-like member may be processed into an orange peel surface provided with many fine irregularities. In this case, an object is brought into contact with the orange peel surface and abraded therewith, so that the object can be cut effectively.

Note that, in the present invention, the entire surface of the wire-like member may be uneven. Alternatively, the surface of the wire-like member may be uneven at least in the portions to be the cutting regions of the wire-like member, and the rest may be even, as described above.

### Seventh Embodiment

The suction catheter of the present invention can also be modified by employing a cutting portion made of a bellows-shaped body instead of the cutting portion made of the spiral body (coil-spring shape) of any one of the above-described first to fifth embodiments. In other words, a seventh embodiment differs from the above-described first to fifth embodiments in terms of the structure of the cutting portion.

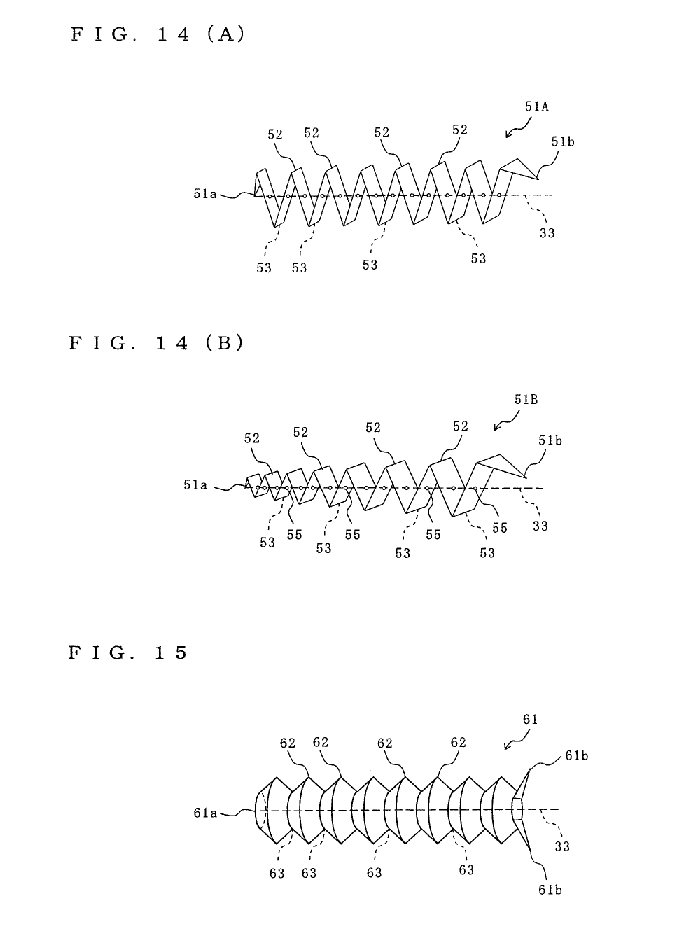

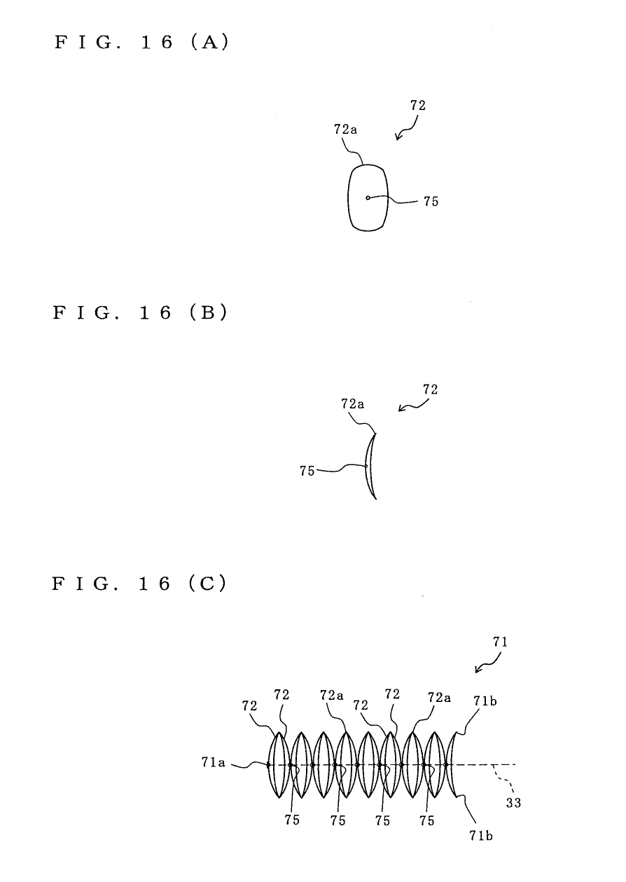

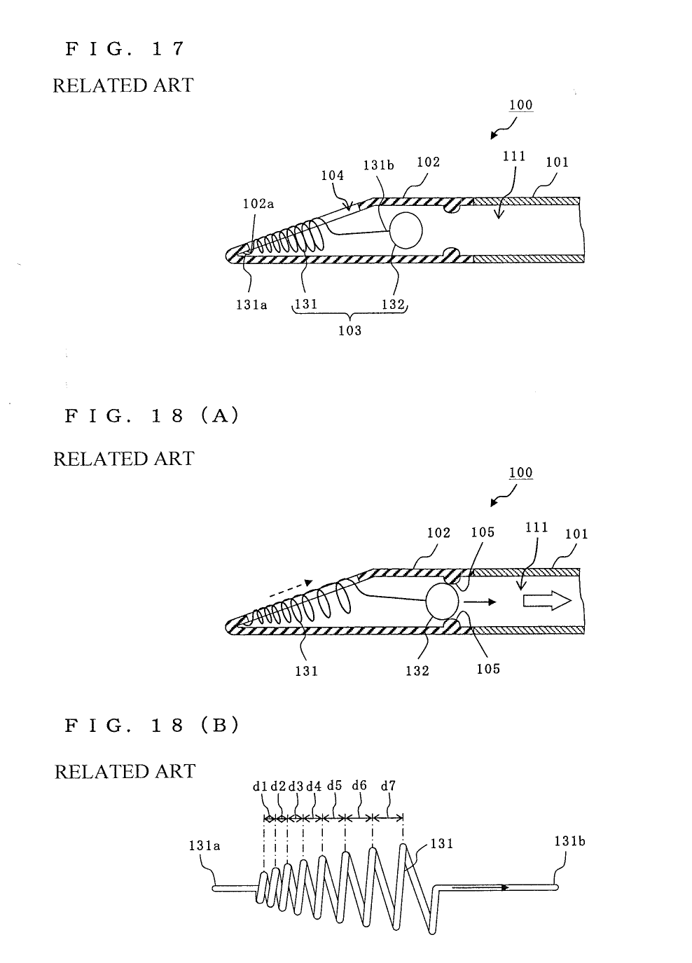

The cutting portion of this embodiment may be made of a bellows-shaped body having mountain fold portions and valley fold portions repeatedly formed therein. For example, the cutting portion may be made of a bellows-shaped plate obtained by bending a sheet-like member to repeatedly form mountain fold portions and valley fold portions, as shown in FIG. 14. Alternatively, the cutting portion may be made of a bellows-shaped cylindrical tube or a bellows-shaped polygonal tube that is a tubular member having a circular or polygonal cross-sectional shape and having mountain fold portions and valley fold portions repeatedly formed therein, as shown in FIG. 15 or 16.

Specifically, FIG. 14A shows a cutting portion 51A made of a bellows-shaped plate obtained by repeatedly forming mountain fold portions 52 and valley fold portions 53 in a sheet-like member. FIG. 14B shows a cutting portion 51B made of a bellows-shaped plate that is obtained by repeatedly forming mountain fold portions 52 and valley fold portions 53 in a sheet-like member, and that has a width or diameter gradually increasing from a first end 51a side to a second end 51b side.

In each of the cutting portions 51A and 51B, the mountain fold portions 52 serves as the object cutting regions, and the mountain fold portions 52 are exposed through the opening portion in the leading-end case portion of the suction catheter. Each of the cutting portions 51A and 51B is disposed in the leading-end case portion to be movable in the axial direction.

As in the case of the object cutting means of each of the above-described first to fifth embodiments, each of the cutting portions 51A and 51B constitutes object cutting means together with a power receiving portion that moves the cutting portion 51A or 51B upon reception of power from the outside and a power transmission rod 33 that connects the cutting portion 51A or 51B to the power receiving portion and that transmits the power received by the power receiving portion to the cutting portion 51A or 51B. Each of the cutting portions 51A and 51B has a first end 51a connected to the power transmission rod 33 and a second end 51b fixed to an internal wall surface of the leading-end case portion on the base-end side thereof.

Note that the bellows-shaped plate has a hole portion 55 at a substantially center of each strip of the folded sheet-like member. The power transmission rod 33 is inserted through the hole portions 55. Accordingly, the bellows-shaped plate extends and contracts along with the motion of the power transmission rod 33 inserted through the hole portions 55.

In each of the cutting portions 51A and 51B made of such a bellows-shaped plate, both the mountain fold portions 52 and the valley fold portions 53 have spring resilience. When the intervals of the surfaces of the folded sheet-like member are reduced, the spring resilience acts to increase the intervals of the surfaces of the folded sheet-like member to restore the original state. In addition, the mountain fold portions 52 serve as cutting regions, so that an object that sticks to an internal wall of a blood vessel can be easily cut.

Meanwhile, FIG. 15 shows a cutting portion 61 made of a bellows-shaped cylindrical tube that is a tubular member having mountain fold portions 62 and valley fold portions 63 repeatedly formed therein.

In this cutting portion 61, the mountain fold portions 62 serve as the object cutting regions, and the mountain fold portions 62 are exposed through an opening portion of a leading-end case portion of the suction catheter. This cutting portion 61 is disposed in the leading-end case portion to be movable in the axial direction.

As in the case of the object cutting means of the above-described first to fifth embodiments, the cutting portion 61 constitutes object cutting means together with a power receiving portion that moves the cutting portion 61 upon reception of power from the outside, and a power transmission rod 33 that connects the cutting portion 61 to the power receiving portion and that transmits the power received by the power receiving portion to the cutting portion 61. The cutting portion 61 has a first end 61a connected to the power transmission rod 33 and a second end 61b fixed to the internal wall surface of the leading-end case portion on the base-end side thereof.

Note that the bellows-shaped tube is hollow, and extends and contracts along with the motion of the power transmission rod 33 inserted into the inside of the bellows-shaped tube.

Although illustration is omitted, the cutting portion 61 made of such a bellows-shaped cylindrical tube may be modified to a bellows-shaped cone or bellows-shaped pyramid having a general outer diameter gradually increasing from a first end 61a side to a second end 61b side.

Accordingly, as in the case of a cutting portion made of a bellows-shaped plate, both the mountain fold portions 62 and the valley fold portions 63 have spring resilience, also when the cutting portion 61 is made of a bellows-shaped cylindrical tube or a bellows-shaped polygonal tube. When the intervals of the surfaces of the folded bellows-shaped tube are reduced, the spring resilience acts to increase the intervals of the surfaces of the folded bellows-shaped tube to restore the original state. In addition, the mountain fold portions 62 serves as cutting regions, so that an object that sticks to an internal wall of a blood vessel can be easily cut.

Moreover, when the cutting portion is made of a bellows-shaped cone or a bellows-shaped pyramid, the degree of the change in the intervals of the mountain fold portions serving as the cutting regions is remarkably different between the first end side where the outer diameter is smaller and the second end side where the outer diameter is larger. However, since the power transmitted from the power receiving portion is received on the first end side where the leading-end portion of the suction catheter is present, the intervals of the cutting regions on the first end side can be changed efficiently and preferentially, so that an object that sticks to an internal wall of a blood vessel can be cut.

Moreover, FIGS. 16A to 16C show a cutting portion 71 made of a bellows-shaped body using multiple leaf spring members 72.

As shown in FIGS. 16A and 16B, each of the leaf spring members 72 has an arch shape formed by curving a planar member in the tip-to-base direction in a side view, and has a hole portion 75, through which the power transmission rod is inserted, near the center thereof in a front view. Accordingly, the leaf spring member 72 has spring resilience that acts to restore the original curved arch shape in a side view, when the leaf spring member 72 is extended to be close to a straight line shape in a side view.

In addition, as shown in FIG. 16C, the cutting portion 71 is formed by stacking multiple leaf spring members 72 that face alternately in opposite directions, and joining the contact edge portions of the leaf spring members 72 together. Accordingly, the spring resilience of the cutting portion 71 is exerted in response to the change in the degree of the curving of the joined multiple leaf spring members 72. Specifically, when the interval between each pair of the leaf spring members 72 is reduced, the curved leaf spring members 72 extend to have a shape similar to a straight line in a side view. Here, to restore their original curved state, the spring resilience of the leaf spring members 72 acts to increase the interval between each pair of the leaf spring members 72.

In the cutting portion 71, an upper edge portion 72a at which each pair of the leaf spring members 72 are connected to each other serves as an object cutting region, and the upper edge portions 72a are exposed through the opening portion of the leading-end case portion of the suction catheter. The cutting portion 71 is disposed in the leading-end case portion to be movable in the axial direction.

As in the case of the object cutting means of the above-described first to fifth embodiments, the cutting portion 71 constitutes object cutting means together with a power receiving portion that moves the cutting portion 71 upon reception of power from the outside, and a power transmission rod 33 that connects the cutting portion 71 to the power receiving portion and that transmits the power received by the power receiving portion to the cutting portion 71. The cutting portion 71 has a first end 71a connected to the power transmission rod 33 and a second end 71b fixed to the internal wall surface of the leading-end case portion on the base-end side thereof.

Accordingly, in the cutting portion made of the bellows-shaped body using the multiple leaf spring members 72, the spring resilience is secured by the leaf spring members 72, and the upper edge portions serve as the cutting regions, so that an object that sticks to an internal wall of a blood vessel can be easily cut.

The present invention is industrially useful in an industry that employs suction catheters used in a treatment where a suction catheter is inserted into a blood vessel to suck and remove an object such as a blood clot therefrom. The present invention is particularly useful in the market of suction catheters for sucking and removing blood clots formed in the arteries of the brain or the heart as well as in the arteries and the veins of the four limbs.

1. A suction catheter comprising:
a catheter main body made of a flexible lengthy body with a lumen formed therein;
a leading-end case portion positioned at a leading-end portion of the catheter main body and having an opening portion for retrieving an object; and
object cutting means that is partially exposed through the opening portion of the leading-end case portion and is movable in an axial direction within the leading-end case portion,
the suction catheter being adapted to be used in a treatment where a leading-end side of the catheter main body is inserted into a blood vessel together with the leading-end case portion, a base-end portion of the catheter main body is connected to a suction apparatus, and the lumen is vacuumed by means of the suction apparatus, so that the object cutting means is operated to cut an object, which sticks to an internal wall of the blood vessel, at the opening portion of the leading-end case portion, and also to suck and retrieve the object through the opening portion into the catheter main body, wherein
the object cutting means includes
a cutting portion that includes a plurality of lines of cutting regions each extending in a substantially perpendicular direction to the axial direction and that has a resilience to restore changed intervals of adjacent ones of the cutting regions,
a power receiving portion that is movable in the axial direction upon reception of power from the outside, and
a power transmission rod that connects the cutting portion to the power receiving portion and that transmits the power received by the power receiving portion to the cutting portion, and
the cutting portion has a first end connected to the power transmission rod on the leading-end side of the leading-end case portion, and a second end fixed to an internal wall surface of the leading-end case portion on a base-end side thereof.

2. The suction catheter according to claim 1, wherein
the cutting portion is a spiral body formed by winding up a wire-like member a number of times.

3. The suction catheter according to claim 2, wherein
the wire-like member has uneven surfaces at least in the cutting regions.

4. The suction catheter according to claim 2, wherein
the wire-like member has a polygonal cross-sectional shape.

5. The suction catheter according to claim 2, wherein
the cutting portion has a width or diameter gradually increasing from the leading-end side to the base-end side.

6. The auction catheter according to claim 3 or 4, wherein
the cutting portion is the spiral body formed by winding up the wire-like member a number of times, and has a width or diameter gradually increasing from the leading-end side to the base-end side.

7. The suction catheter according to claim 1, wherein
the cutting portion is a bellows-shaped body including a sheet-like member or tubular member having mountain fold portions and valley fold portions repeatedly formed therein.

8. The suction catheter according to claim 7, wherein
the cutting portion has a width or diameter gradually increasing from the leading-end side to the base-end side.

9. The suction catheter according to claim 1, wherein
the leading-end case portion includes a locking stopper formed in an internal wall surface of the leading-end case portion, the locking stopper restricting a movement of the object cutting means toward the base-end portion of the catheter main body.

10. The suction catheter according to claim 2 or 7, wherein
the leading-end case portion includes a locking stopper formed in an internal wall surface of the leading-end case portion, the locking stopper restricting a movement of the object cutting means toward the base-end portion of the catheter main body.

11. The suction catheter according to claim 1, wherein
at least a part of any one of the leading-end case portion and the object cutting means is made of a metal member.

12. The suction catheter according to claim 2 or 7, wherein
at least a part of any one of the leading-end case portion and the object cutting means is made of a metal member.

13. The suction catheter according to claim 1, wherein
the opening portion is provided in a leading-end inclined face of an obliquely-formed leading-end portion of the leading-end case portion.

14. The suction catheter according to claim 2 or 7, wherein
the opening portion is provided in a leading-end inclined face of an obliquely-formed leading-end portion of the leading-end case portion.

15. The suction catheter according to claim 1, wherein
the opening portion is provided in a leading-end side circumferential-body surface of the leading-end case portion.

16. The suction catheter according to claim 2 or 7, wherein
the opening portion is provided in a leading-end side circumferential-body surface of the leading-end case portion.

17. The suction catheter according to claim 1, wherein
the power receiving portion includes a magnetic material, and
the leading-end case portion includes magnetic-force generating means provided to the internal wall surface of the leading-end case portion and configured to supply power to the power receiving portion.

18. The suction catheter according to claim 2 or 7, wherein
the power receiving portion includes a magnetic material, and
the leading-end case portion includes magnetic-force generating means provided to the internal wall surface of the leading-end case portion and configured to supply power to the power receiving portion.

19. The suction catheter according to claim 1, wherein
at least one of the catheter main body and the leading-end case portion includes a guide-wire hole formed in an outer surface thereof, the guide-wire hole allowing a guide-wire to be inserted thereinto.

20. The suction catheter according to claim 2 or 7, wherein
at least one of the catheter main body and the leading-end case portion includes a guide-wire hole formed in an outer surface thereof, the guide-wire hole allowing a guide-wire to be inserted thereinto.
